(19) United States
(12) Patent Application Publication
(10) Pub. No.: US 2004/0014768 A1
(43) Pub. Date: Jan. 22, 2004

(54) Chromenone derivatives and their use for treating diseases in conjunction with 5-hta1receptors and/or dopamine d2 receptors

(76) Inventors: Rudolf Gottschlich, Reinheim (DE); Karl-Augst Ackermann, Ober-Ramstadt (DE); Helmut Prücher, Heppenheim (DE); Christoph Seyfried, Seeheim (DE); Gerd Bartoszyk, Weiterstadt (DE); Christoph Van Amsterdam, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/362,236

(22) PCT Filed: Jul. 24, 2001
(86) PCT No.: PCT/EP01/08528

(30) Foreign Application Priority Data
Aug. 24, 2000 (DE) 10041574.1

(51) Int. Cl. A61K 31/496; C 07D 4 5/02; C 07D 4 5/14

(52) U.S. Cl. 514/253.06; 514/254.09; 544/363; 544/373

(57) ABSTRACT

Novel chromenone derivatives of the formula I

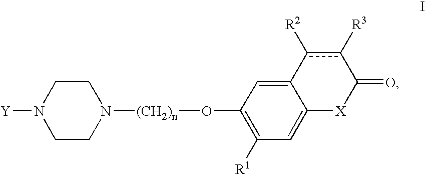

in which Y, X, R¹, R², R³ and n are as defined in claim 1, as inhibitors of the 5-HT_{1A} receptor and of the dopamine D₂ receptor.

The invention relates to chromenone derivatives of the formula I

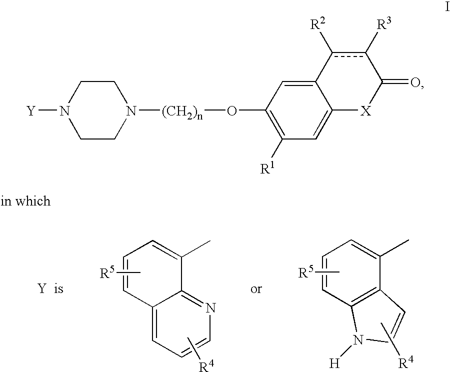

X is O or NR⁴,

[symbol] is a single or double bond,

R¹ is H, A, OA or Hal,

R², R³ and R⁴ are each, independently of one another, H or A,

R⁵ is H or Hal,

A is alkyl having 1 to 6 carbon atoms,

Hal is F, Cl, Br or I, and

n is 2, 3, 4 or 5,

and their physiologically acceptable salts and solvates.

Partially similar compounds are disclosed in EP 0 584 091.

The invention had the object of finding novel compounds having valuable properties, in particular those which can be used for the production of medicaments.

Psychoses, which also include illnesses from the schizophrenia group, are attributed to overactivity of the limbic dopamine system (Snyder et al., Science 184: 1243-1253, 1974). The antipsychotic effect of neuroleptics is attributed to their D₂-antagonistic properties (regarding the nomenclature of the receptors: Basic Neurochemistry, Editors: G. J. Siegel, B. W. Agranoff, R. W. Albers, P. B. Molinoff, 5th Edition, Raven Press, Ltd., N.Y. USA, Chapters 12 and 13; also the following papers: Creese et al., Science 192: 481-483, 1976; Farde et al., Psychopharmacology 99: 28-31, 1989; Feeman et al., Nature 261: 717-719, 1976; Wiesel et al., Prog. Neuro-Psychopharmacol. & Biol. Psychiat. 14: 759-767, 1990). The classical dopamine hypothesis of schizophrenia states that neuroleptics have to bind to the D₂ receptor.

The use of classical D₂ antagonists is greatly restricted owing to their extrapyramidal side-effects, especially in the case of chronic administration. The extrapyramidal side-effects include, for example, tremor, akinesia, dystonia and acathisia (Cavallaro & Smeraldi, CNS Drugs 4: 278-293, 1995). There are only few antipsychotics which cause significantly fewer or no extrapyramidal side-effects at all; these are known as atypical neuroleptics (Kervin, Brit. J. Psychiatry 1964, 141-148, 1994). The prototypical atypical neuroleptic clozapine has extremely low extrapyramidal side-effects, but causes other severe complications, such as occasionally fatal agranulocytosis (Alvir et al., New Engl. J. Med. 329: 162-167, 1993).

Since 5-HT_{1A} agonists augment antipsychotic properties of conventional dopamine D₂ antagonists in animals (Wadenberg & Ahlenios, J. Neural. Transm. 74: 195-198, 1988) and prevent catalepsy induced by dopamine D₂ antagonists (Costall et al., Neuropharmacology 14: 859-868, 1975), 5-HT_{1A}-agonistic properties may be advantageous. The efficacy of buspirone, a drug having 5-HT_{1A}-agonistic and dopamine D₂-antagonistic properties, has been demonstrated in schizophrenia patients (Goff et al., J. Clin, Psychopharmacol. 11: 193-197, 1991). Apart from various dopamine autoreceptor agonists which also have significant affinity to the 5-HT_{1A} receptor (for example U-86170F, Lahti et al., Naunyn-Schmiedeberg's Arch. Pharmacol. 344: 509-513, 1991), PD1431188 (Melzer et al., J. Pharmacol. Exp. Ther. 274: 912-920, 1995) and roxindole (Bartoszyk et al., J. Pharmacol., Exp. Ther. 276: 41-48, 1996), only few dopamine D₂ antagonists have been developed which also have affinity to the 5-HT_{1A} receptor, such as mazapertine (Reiz et al., J. Mid. Chem. 37: 1060-1062, 1994), S16924 (Millan et al., Br. J. Pharmacol. 114: 156 B, 1995) or ziprasidone (Seeger et al., J. Pharmacol. Exp. Ther. 275: 101-113, 1995). These previously known compounds have disadvantages with respect to affinity or specificity. Thus, mazapertine also exhibits affinity for the α₁ receptor. S16924 additionally has 5-HT_{2A/C}-antagonistic properties, and ziprasidone also binds to the 5-HT_{1D/2A/2C} receptors.

It has been found that the compounds of the formula I and their salts have very valuable pharmacological properties and are well tolerated. They act in particular on the central nervous system. In particular, they have high affinity to receptors of the 5-HT_{1A} type and/or of the dopamine D₂ type. Compounds of the formula I are particularly preferably at the same time agonists of the 5-HT_{1A} receptor and antagonists of the D₂ receptor. Binding to additional 5-HT_{1D/2A/2C} receptors is not observed.

The binding properties of the compounds of the formula I can be determined by known 5-HT_{1A} (serotonin) binding tests and dopamine binding tests (5-HT_{1A} (serotonin) binding test: Matzen et al., J. Med. Chem., 43, 1149-1157, (2000) in particular page 1156 with reference to Eur. J. Pharmacol.: 140, 143-155 (1987); dopamine binding tests: Böttcher et al., J. Med. Chem.: 35, 4020-4026, (1992) with reference to J. Neurochem.: 46, 1058-1067 (1986)).

The compounds according to the invention can be employed for the treatment of illnesses which are associated with the serotonin and dopamine neurotransmitter system and in which high-affinity serotonin receptors (5-HT_{1A} receptors) and/or dopamine D₂ receptors are involved.

The most important indication for the administration of the compound of the general formula I are psychoses of all types, in particular mental illnesses from the schizophrenia group. In addition, the compounds can also be employed for reducing defects in cognitive ability, i.e. for improving learning ability and memory. The compounds of the general formula I are also suitable for combating the symptoms of Alzheimer's disease. In addition, the substances of the general formula I according to the invention are suitable for the prophylaxis and control of cerebral infarctions (apoplexia cerebri), such as cerebral strokes and cerebral ischaemia. The substances are furthermore used for the treatment of illnesses such as pathological states of anxiety, overexcitation, hyperactivity and attention disorders in children and youths, severe development disorders and disorders of social behaviour with mental retardation, depression, obsessive-compulsive disorders in the narrower (OCD) and broader sense (OCSD), certain disorders of sexual function, sleep disturbances and disorders in nutrient take-up, as well as psychiatric symptoms in age dementia and dementia of the Alzheimer's type, i.e. illnesses of the central nervous system in the broadest sense.

The compounds of the formula I are likewise suitable for the treatment of extrapyramidal motor diseases, for the treatment of side-effects which occur in the treatment of extrapyramidal motor diseases with conventional anti-Parkinson's medicaments, of the treatment of extrapyramidal symptoms (EPS) induced by neuroleptics.

Extrapyramidal motor diseases are, for example, idiopathic Parkinson's disease, parkinsonian syndrome, dyskinetic choreatic or dystonic syndrome, tremor, Gilles de la Torette syndrome, ballism, muscle cramps, restless legs syndrome or Wilson's disease.

The compounds of the formula I are particularly suitable for the treatment of side-effects which occur in the treatment of idiopathic Parkinson's disease with conventional Parkinson's medicaments. They can therefore also be used as add-on therapy in the treatment of Parkinson's disease. Known Parkinson's medicaments are drugs such as L-dopa (levodopa) and L-dopa combined with benserazide or carbidopa, dopamine agonists, such as bromocriptine, apomorphine, cabergoline, pramipexole, ropinirole, pergolide, dihydro-α-ergocriptine or lisuride, and all medicaments which effect stimulation of the dopamine receptor, inhibitors of catechol O-methyl transferase (COMT), such as entacapone or tolcapone, inhibitors of monoamine oxidase (MAO), such as selegiline, and antagonists of N-methyl-D-aspartate (NMDA) receptors, such as amantadine or budipine.

The compounds of the general formula I and their tolerated salts and solvates can thus be employed as active ingredients for medicaments, such as anxiolytics, antidepressives, neuroleptics and/or antihypertensives.

A measure of the take-up of a medicament active ingredient in an organism is its bioavailability.

If the medicament active ingredient is administered intravenously to the organism in the form of an injection solution, its absolute bioavailability, i.e. the fraction of the drug which reaches the systemic blood, i.e. the general circulation, in unchanged form is 100%.

In the case of oral administration of a therapeutic active ingredient, the active ingredient is generally in the form of a solid in the formulation and must therefore first be dissolved so that it is able to overcome the entry barriers, for example the gastrointestinal tract, the oral mucous membrane, nasal membranes or the skin, in particular the stratum corneum, or can be absorbed by the body. Pharmacokinetic data, i.e. on the bioavailability, can be obtained analogously to the method of J. Shaffer et al., J. Pharm. Sciences, 1999, 88, 313-318.

A further measure of the absorbability of a therapeutic active ingredient is the logD value, since this value is a measure of the lipophilicity of a molecule.

If the compounds of the general formula I are optically active, the formula I covers both all isolated optical antipodes and the corresponding possibly racemic mixtures in any conceivable composition.

The term “solvates of the compounds of the formula I” is taken to mean adducts of inert solvent molecules onto the compounds of the formula I which form owing to their mutual attractive force. Solvates are, for example, monohydrates or dihydrates or addition compounds with alcohols, such as, for example, with methanol or ethanol.

The invention relates to the compounds of the formula I and their salts and solvates according to claim 1, and to a process for the preparation of compounds of the formula I and their salts and solvates, characterised in that

(a) a compound of the formula II

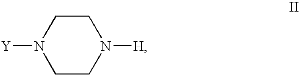

in which Y is as defined in claim 1,

is reacted with a compound of the formula III

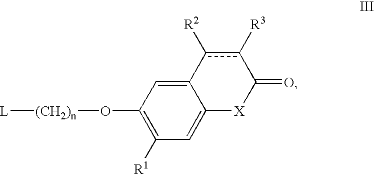

in which R¹, R², R³ and n are as defined in Claim 1,

[symbol] is a single or double bond, and

L is Cl, Br or a reactive esterified OH group, or

(b) a compound of the formula IV

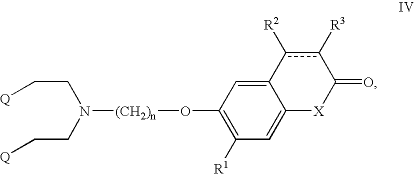

in which R¹, R², R³ and n are as defined in claim 1,

[symbol] is a single or double bond, and

Q is Cl or Br,

is reacted with a compound of the formula V

Y—NH₂ V

in which Y is as defined in claim 1, or

(c) a compound of the formula VI

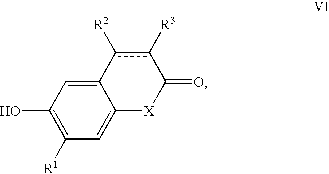

in which R¹, R² and R³ are as defined in claim 1, and

[symbol] is a single or double bond,

is reacted with a compound of the formula VII

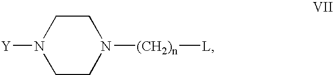

in which Y and n are as defined in claim 1, and

L is Cl, Br or a reactive esterified OH group, and/or a basic or acidic compound of the formula I is converted into one of its salts or solvates by treatment with an acid or base.

In the above formulae, A is alkyl, is linear or branched, and has 1 to 6, preferably 1, 2 or 3 carbon atoms. A is preferably methyl, ethyl, n-propyl, isopropyl, n-butyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1,1,2- or 1,2,2-trimethylpropyl. A is preferably methyl, ethyl, n-propyl or isopropyl. A is particularly preferably methyl.

OA is alkoxy, where A is as defined above.

Hal is preferably F, Cl or bromine.

The representation

[symbol] in the formulae I, III, IV and VI denotes that the bond marked in this way may be either a single bond or a double bond. The bond marked in this way is particularly preferably a double bond.

Y is quinolin-8-yl or 1H-indol-4-yl, it being possible for both heterocycles to be substituted by R⁴, where R⁴ is as defined below. The substituent R⁴ is preferably in the 2-position of the heterocycle.

Y is particularly preferably 2-methylquinolin-8-yl, quinolin-8-yl or 1H-indol-4-yl.

X is O or NR², where R² is as defined below. X is particularly preferably O.

R¹ is H, A, OA or Hal, where A and Hal are as defined above. R¹ is particularly preferably OA.

R², R³ and R⁴ are each, independently of one another, H or A, where A is as defined above.

R² is particularly preferably A.

R³ is particularly preferably A.

R⁵ is H or Hal, where Hal is as defined above. R⁵ is particularly preferably H.

n is preferably 2, 3, 4 or 5. n is particularly preferably 2 or 3.

Accordingly, the invention relates in particular to the compounds of the formula I in which at least one of the said radicals has one of the preferred meanings indicated above. Some preferred groups of compounds may be expressed by the following sub-formulae Ia to I3, which conform to the formula I and in which the radicals not denoted in greater detail are as defined under the formula I, but in which

in Ia X is O;

in Ib the marked bond [symbol] is a double bond;

in Ic n is 2 or 3;

in Id X is O,

the marked bond [symbol] is a double bond, and

n is 3, or

in Ie X is O,

the marked bond [symbol] is a double bond, and

n is 2.

The compounds of the formula I according to claim 1 and also the starting materials for their preparation are, in addition, prepared by methods known per se, as described in the literature (for example in the standard works, such as Houben-Weyl, Methoden der Organischen Chemie [Methods of Organic Chemistry], Georg-Thieme Verlag, Stuttgart), to be precise under reaction conditions which are known and suitable for the said reactions. Use can also be made here of variants which are known per se, but are not explained here in greater detail.

If desired, the starting materials can also be formed in situ, so that they are not isolated from the reaction mixture, but instead immediately converted further into the compounds of the formula I according to claim 1.

The starting compounds of the formulae II, III, IV, V, VI and VII are generally known. If they are novel, they can, however, be prepared by methods known per se.

Compounds of the formula I can be prepared by nucleophilic substitution of the leaving group L of the compounds of the formula III, where L is Cl, Br or a reactive esterified OH group, by the piperazine nitrogen of the compound of the formula II under standard conditions. Reactive esterified OH groups are esterified, for example, with alkylsulfonic acids or arylsulfonic acids, meaning that, for example, mesylates or tosylates of the compounds of the formula III are suitable.

Compounds of the formula I can likewise be prepared by reaction of the dihalide of the formula IV with an amine of the formula V.

Compounds of the formula I may furthermore be prepared by reaction of the alcohol of the formula VI with a compound of the formula VII in which L is Cl, Br or a reactive esterified OH group. Reactive esterified OH groups are, for example, hydroxyl groups which have been esterified with alkylsulfonic acids or arylsulfonic acids, meaning that mesylates or tosylates of the compounds of the formula VII are suitable.

The reaction conditions for nucleophilic substitutions, as described above, are adequately known to the person skilled in the art, see also Organikum [Practical Organic Chemistry], 17th Edition, Deutscher Verlag für Wissenschaften, Berlin, 1988.

A base of the formula I can be converted into the associated acid-addition salt using an acid, for example by reaction of equivalent amounts of the base and the acid in an inert solvent, such as ethanol, followed by evaporation. Suitable acids for this reaction are, in particular, those which give physiologically acceptable salts. Thus, it is possible to use inorganic acids, for example sulfuric acid, sulfurous acid, dithionic acid, nitric acid, hydrohalic acids, such as hydrochloric acid or hydrobromic acid, phosphoric acids, such as, for example, orthophosphoric acid, or sulfamic acid, furthermore organic acids, in particular aliphatic, alicyclic, araliphatic, aromatic or heterocyclic monobasic or polybasic carboxylic, sulfonic or sulfuric acids, for example formic acid, acetic acid, propionic acid, hexanoic acid, octanoic acid, decanoic acid, hexadecanoic acid, octadecanoic acid, pivalic acid, diethylacetic acid, malonic acid, succinic acid, pimelic acid, fumaric acid, maleic acid, lactic acid, tartaric acid, malic acid, citric acid, gluconic acid, ascorbic acid, nicotinic acid, isonicotinic acid, methane- or ethanesulfonic acid, benzenesulfonic acid, trimethoxybenzoic acid, adamantanecarboxylic acid, p-toluenesulfonic acid, glycolic acid, embonic acid, chlorophenoxyacetic acid, aspartic acid, glutamic acid, proline, glyoxylic acid, palmitic acid, paraa-chlorophenoxyisobutyric acid, cyclohexanecarboxylic acid, glucose 1-phosphate, naphthalenemono- and -disulfonic acids, or laurylsulfuric acid. Salts with physiologically unacceptable acids, for example picrates, can be used for the isolation and/or purification of the compounds of the formula I. On the other hand, compounds of the formula I can be converted using bases (for example sodium hydroxide, potassium hydroxide, sodium carbonate, potassium carbonate) into the corresponding metal salts, in particular alkali metal salts or alkaline-earth metal salts, or into the corresponding ammonium salts.

The invention also relates to the compounds of the formula I according to claim 1 and their physiologically acceptable salts or solvates as medicament active ingredients.

The invention furthermore relates to compounds of the formula I according to claim 1 and their physiologically acceptable salts or solvates as D₂ receptor antagonists and 5HT_{1A} agonists.

The invention also relates to the compounds of the formula I according to claim 1 and their physiologically acceptable salts or solvates for use in combating illnesses.

The invention furthermore relates to pharmaceutical preparations comprising at least one compound of the formula I and/or one of its physiologically acceptable salts or solvates which are prepared, in particular, by non-chemical methods. In this case, the compounds of the formula I can be converted into a suitable dosage form together with at least one solid, liquid and/or semiliquid excipient or assistant and optionally in combination with one or more further active ingredients.

These preparations can be used as medicaments in human or veterinary medicine. Suitable excipients are organic or inorganic substances which are suitable for enteral (for example oral), parenteral or topical administration and do no react with the novel compounds, for example water, vegetable oils, benzyl alcohols, alkylene glycols, polyethylene glycols, glycerol triacetate, gelatine, carbohydrates, such as lactose or starch, magnesium stearate, talc or Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The novel compounds may also be lyophilised and the resultant lyophilisates used, for example, for the preparation of injection preparations. The preparations indicated may be sterilised and/or comprise assistants, such as lubricants, preservatives, stabilisers and/or wetting agents, emulsifiers, salts for modifying the osmotic pressure, buffer substances, colorants, flavours and/or a plurality of further active ingredients, for example one or more vitamins.

The invention furthermore relates to the use of a compound of the general formula I or one of its tolerated salts or solvates for the preparation of a medicament which is suitable for the treatment of human or animal illnesses, in particular illnesses of the central nervous system, such as pathological states of stress, depression and/or psychoses, for reducing side-effects in the treatment of high blood pressure (for example with a-methyldopa), for the treatment of endoclinological and/or gynaecological illnesses, for example for the treatment of agromegaly, hypogonadism, secondary amenorrhoea, post-menstrual syndrome and undesired lactation in puberty and for the prophylaxis and therapy of cerebral illnesses (for example migraine), in particular in geriatrics, in a similar manner as certain Ergot alkaloids, and for the control and prophylaxis of cerebral infarction (apoplexia cerebri), such as cerebral strokes and cerebral ischaemia, for the treatment of extrapyramidal motor diseases, for the treatment of side effects which occur in the treatment of extrapyramidal motor diseases with conventional anti-Parkinson's medicaments, or for the treatment of extrapyramidal symptoms (EPS) induced by neuroleptics. In addition, the pharmaceutical preparations and medicaments which comprise a compound of the general formula I are suitable for improving cognitive ability and for the treatment of the symptoms of Alzheimer's disease. In particular, medicaments of this type are suitable for the treatment of mental illnesses from the schizophrenia group and for combating psychotic anxiety states. For the purposes of the invention, the term treatment includes the prophylaxis and therapy of human or animal illnesses.

The substances of the general formula I are normally administered analogously to known, commercially available pharmaceutical preparations (for example bromocriptine and dihydroergocornine), preferably in doses of between 0.2 and 500 mg, in particular between 0.2 and 15 mg per dosage unit. The daily dosage unit is between 0.001 and 10 mg per kg of body weight. Low doses (of between 0.2 and 1 mg per dosage unit, from 0.001 to 0.005 mg per kg of body weight) are particularly suitable for pharmaceutical preparations for the treatment of migraine. A dose of between 10 and 50 mg per dosage unit is preferred for other indications. However, the dose to be administered depends on a multiplicity of factors, for example on the efficacy of the corresponding component, the age, the body weight and the general state of health of the patient.

Above and below, all temperatures are given in ° C. In the following examples, “conventional work-up” means that water is added if necessary, the pH is, if necessary, adjusted to between 2 and 10, depending on the constitution of the end product, the mixture is extracted with ethyl acetate or dichloromethane, the phases are separated, the organic phase is dried over sodium sulfate and evaporated, and the product is purified by chromatography on silica gel, by preparative HPLC and/or by crystallisation. The purified compounds are, if desired, freeze-dried.

### EXAMPLE 1

2.5 g of 2-methyl-8-piperazin-1-ylquinoline hydrochloride, 2.6 g of potassium carbonate and 1 g of potassium iodide are added under inert-gas conditions to a solution of 3 g of 6-(3-bromopropoxy)-7-methoxy-3,4-dimethylchromen-2-one in 150 ml of dimethylformamide, and the mixture is heated at 80° C. for 48 hours. Conventional work-up gives 7-methoxy-3,4-dimethyl-6-{3-[4-(2-methylquinolin-8-yl)piperazin-1-yl]propoxy)chromen-2-one.

### EXAMPLE 2

Analogously to Example 1, the reaction of 6-(3-chloropropoxy)-7-methoxy-3,4-dimethylchromen-2-one with 4-piperazin-1-yl-1H-indole dihydrochloride gives 6-{3-[4-(1H-indol-4-yl)piperazin-1-yl]propoxy}-7-methoxy-3,4-di-methylchromen-1-one.

### EXAMPLE 3

0.66 g of 4-piperazin-1-yl-1H-indol and 0.84 g of sodium hydrogencarbonate are added to a solution of 1 g of ethyl 2-(7-methoxy-3,4-dimethyl-2-oxo-2H-chromen-6-yloxy)methanesul fonate in 30 ml of acetonitrile, and the mixture is refluxed for 25 hours. After the mixture has been cooled to room temperature, it is poured onto ice and subjected to conventional work-up, giving 6-{2-[4-(1H-indol-4-yl)piperazin-1-yl]ethoxy}-7-methoxy-3,4-dimethylchromen-2-one; m.p. 201-202° C.

In order to precipitate the salt, 6-{2-[4-(1H-indol-4-yl)piperazin-1-yl]ethoxy}-7-methoxy-3,4-dimethylchromen-2-one is suspended in isopropanol, and ethereal hydrochloric acid is added until the mixture is acidic, giving 6-{2-[4-(1H-indol-4-yl)piperazin-1-yl]ethoxy}-7-methoxy-3,4-dimethylchromen-2-one dihydrochloride; m.p. 231-237° C.

### EXAMPLE 4

Analogously to Example 3, the reaction of ethyl 2-(7-methoxy-3,4-dimethyl-2-oxo-2H-chromen-6-yloxy)methanesulfonate with

8-piperazin-1-ylquinoline gives 7-methoxy-3,4-dimethyl-6-[2-(4-quinolin-8-ylpiperazin-1-yl)ethoxy]chromen-2-one; m.p. 164-165° C.;

after precipitation with ethereal hydrochloric acid, 7-methoxy-3,4-dimethyl-6-[2-(4-quinolin-8-ylpiperazin-1-yl)ethoxy]chromen-2-one dihydrochloride dihydrate; m.p. 225-228° C.;

2-methyl-8-piperazin-1-ylquinoline gives 7-methoxy-3,4-dimethyl-6-{2-[4-(2-methylquinolin-8-yl)piperazin-1-yl]-ethoxy}chromen-2-one; m.p. 168-170° C.;

after precipitation with ethereal hydrochloric acid, 7-methoxy-3,4-dimethyl-6-{2-[4-(2-methylquinolin-8-yl)piperazin-1-yl]-ethoxy}chromen-2-one dihydrochloride monohydrate; m.p. 198-200° C.

### EXAMPLE 5

Analogously to Example 3, the reaction of propyl 3-(7-methoxy-3,4-dimethyl-2-oxo-2H-chromen-6-yloxy)methanesulfonate with

8-piperazin-1-ylquinoline gives 7-methoxy-3,4-dimethyl-6-[3-(4-quinolin-8-ylpiperazin-1-yl)propoxy]-chromen-2-one; m.p. 225-228° C.;

after precipitation with ethereal hydrochloric acid, 7-methoxy-3,4-dimethyl-6-[3-(4-quinolin-8-ylpiperazin-1-yl)propoxy]-chromen-2-one dihydrochloride pentahydrate; m.p. 233-242° C.;

2-methyl-8-piperazin-1-ylquinoline gives 7-methoxy-3,4-dimethyl-6-{3-[4-(2-methylquinolin-8-yl)piperazin-1-yl]-propox}chromen-2-one; m.p. 163-164° C.;

after precipitation with ethereal hydrochloric acid, 7-methoxy-3,4-dimethyl-6-{3-[4-(2-methylquinolin-8-yl)piperazin-1-yl]-propoxy}chromen-2-one dihydrochloride tetrahydrate; m.p. 178-201° C.;

4-piperazin-1-yl-1H-indole gives 6-{3-[4-(1H-indol-4-yl)piperazin-1-yl]propoxy}-7-methoxy-3,4-dimethyl-chromen-2-one; m.p. 193-195° C.;

after precipitation with ethereal hydrochloric acid, 6-{3-[4-(1H-indol-4-yl)piperazin-1-yl]propoxy}-7-methoxy-3,4-dimethyl-chromen-2-one dihydrochloride dihydrate; m.p. 221-224° C.

The examples below relate to pharmaceutical preparations:

### EXAMPLE A

Injection Vials

A solution of 100 g of an active ingredient of the formula I and 5 g of disodium hydrogenphosphate in 3 l of bidistilled water is adjusted to pH 6.5 using 2N hydrochloric acid, sterile filtered, transferred into injection vials, lyophilised under sterile conditions and sealed under sterile conditions. Each injection vial contains 5 mg of active ingredient.

### EXAMPLE B

Suppositories

A mixture of 20 g of an active ingredient of the formula I is melted with 100 g of soya lecithin and 1400 g of cocoa butter, poured into moulds and allowed to cool. Each suppository contains 20 mg of active ingredient.

### EXAMPLE C

Solution

A solution is prepared from 1 g of an active ingredient of the formula I, 9.38 g of NaH₂PO₄. 2 H₂O, 28.48 g of Na₂HPO₄. 12 H₂O and 0.1 g of benzalkonium chloride in 940 ml of bidistilled water. The pH is adjusted to 6.8, and the solution is made up to 1 l and sterilised by irradiation. This solution can be used in the form of eye drops.

### EXAMPLE D

Ointment

500 mg of an active ingredient of the formula I are mixed with 99.5 g of Vaseline under aseptic conditions.

### EXAMPLE E

Tablets

A mixture of 1 kg of active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### EXAMPLE F

Coated Tablets

Tablets are pressed analogously to Example E and subsequently coated in a conventional manner with a coating of sucrose, potato starch, talc, traga-canth and dye.

### EXAMPLE G

Capsules

2 kg of active ingredient of the formula I are introduced into hard gelatine capsules in a conventional manner in such a way that each capsule contains 20 mg of the active ingredient.

### EXAMPLE H

Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

### EXAMPLE I

Inhalation Spray

14 g of active ingredient of the formula I are dissolved in 10 l of isotonic NaCl solution, and the solution is transferred into commercially available spray containers with a pump mechanism. The solution can be sprayed into the mouth or nose. One spray shot (about 0.1 ml) corresponds to a dose of about 0.14 mg.

1. Compounds of the formula I 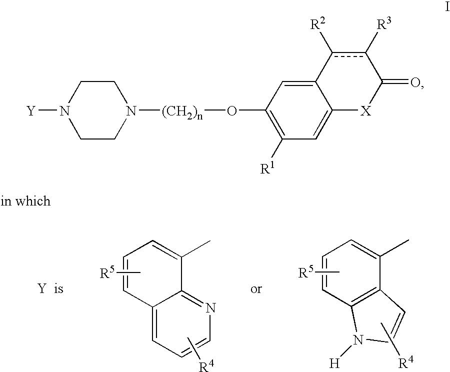 [symbol]
is a single or double bond,
X is O or NR⁴,
R¹ is H, A, OA or Hal,
R², R³ and R⁴ are each, independently of one another, H or A,
R⁵ is H or Hal,
A is alkyl having 1 to 6 carbon atoms,
Hal is F, Cl, Br or I, and
n is 2, 3, 4 or 5,
and their physiologically acceptable salts and solvates:

2. Compounds of the formula I according to claim 1 in which X is O.

3. Compounds of the formula I according to claim 1 or 2, in which the marked bond [symbol] is a double bond.

4. Compounds of the formula I according to one or more of claims 1 to 3 in which n is 3.

5. Compounds of the formula I according to one or more of claims 1 to 3 in which n is 2.

6. Compounds according to claim 1:
a) 7-methoxy-3,4-dimethyl-6-{3-[4-(2-methylquinolin-8-yl)piperazin-1-yl]propoxy}chromen-2-one,
b) 6-(3-[4-(1H-indol-4-yl)piperazin-1-yl]-propoxy}-7-methoxy-3,4-dimethylchromen-2-one,
c) 7-methoxy-3,4-dimethyl-6-[3-(4-quinolin-8-yl)piperazin-1-yl]-propoxy}chromen-2-one,
d) 6-{2-[4-(1H-indol-4-yl)piperazin-1-yl]ethoxy}-7-methoxy-3,4-dimethylchromen-2-one,
e) 7-methoxy-3,4-dimethyl-6-[2-(4-quinolin-8-yl)piperazin-1-yl]-ethoxy}chromen-2-one,
f) 7-methoxy-3,4-dimethyl-6-{2-[4-(2-methylquinolin-8-yl)piperazin-1-yl]ethoxy}chromen-2-one,
and their physiologically acceptable salts and solvates.

7. Process for the preparation of the compounds of the formula I according to claim 1 and their salts and solvates, characterised in that
(a) a compound of the formula II 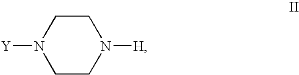
in which Y is as defined in claim 1,
is reacted with a compound of the formula III 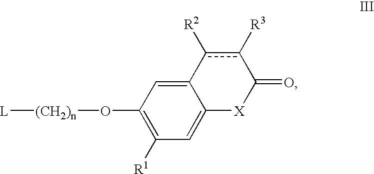
in which R¹, R², R³ and n are as defined in claim 1, [symbol]
is a single or double bond, and
L is Cl, Br or a reactive esterified OH group, or
(b) a compound of the formula IV 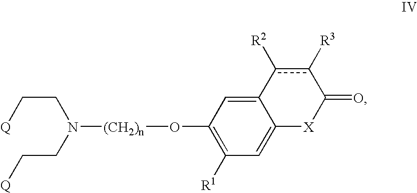
in which R¹, R², R³ and n are as defined in claim 1, [symbol]
is a single or double bond, and
Q is Cl or Br,
is reacted with a compound of the formula V
Y—NH₂ V
in which Y is as defined in claim 1, or
(c) a compound of the formula VI 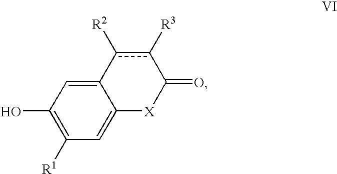
in which R¹, R² and R³ are as defined in claim 1, and [symbol]
is a single or double bond,
is reacted with a compound of the formula VII 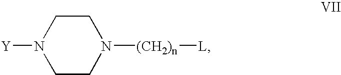
in which Y and n are as defined in claim 1, and
L is Cl, Br or a reactive esterified OH group, and/or a basic or acidic compound of the formula I is converted into one of its salts or solvates by treatment with an acid or base.

8. Compounds of the formula I according to one or more of claims 1 to 6 and their physiologically acceptable salts or solvates as medicament active ingredients.

9. Compounds of the formula I according to one or more of claims 1 to 6 and their physiologically acceptable salts or solvates as D₂ receptor antagonists and 5-HT_{1A} agonists.

10. Pharmaceutical preparation, characterised by a content of at least one compound of the formula I according to one or more of claims 1 to 6 and/or one of its physiologically acceptable salts or solvates.

11. Use of compounds of the formula I according to one or more of claims 1 to 6 and/or their physiologically acceptable salts or solvates for the preparation of a medicament.

12. Use of compounds of the formula I according to one or more of claims 1 to 6 and/or their physiologically acceptable salts or solvates for the preparation of a medicament for combating illnesses which are associated with the serotonin and dopamine neuro-transmitter system and in which high-affinity serotonin receptors (5-HT_{1A} receptors) and/or dopamine D₂ receptors are involved.

13. Use of compounds of the formula I according to one or more of claims 1 to 6 and/or their physiologically acceptable salts or solvates for the preparation of a medicament for combating psychoses, symptoms of Alzheimer's disease, pathological states of anxiety, overexcitement, hyperactivity, attention disorders in children and youths, severe development disorders and disorders of social behaviour with mental retardation, depression, obsessive-compulsive disorders in the narrower (OCD) and broader sense (OCSD), impairment of sexual function, sleep disturbances, disorders in nutrient take-up, and psychiatric symptoms of this type as part of age dementia and dementia of the Alzheimer's type, for reducing defects in cognitive ability, or for the prophylaxis and control of cerebral infarctions (apoplexia cerebri), for the treatment of extrapyramidal motor diseases, for the treatment of side-effects which occur in the treatment of extrapyramidal motor diseases with conventional anti-Parkinson's medicaments, or for the treatment of extrapyramidal symptoms (EPS) induced by neuroleptics.

14. Use of compounds of the formula I according to one or more of claims 1 to 6 and/or their physiologically acceptable salts or solvates for the preparation of a medicament for combating schizophrenia.

9. (Amended) Compounds of the formula I according to claim 1 and their physiologically acceptable salts or solvates as D₂ receptor antagonists and 5-HT_{1A} agonists. 10. (Amended) Pharmaceutical preparation, characterized by a content of at least one compound of the formula I according to claim 1 and/or one of its physiologically acceptable salts or solvates. 11. (Amended) Use of compounds of the formula I according to claim 1 and/or their physiologically acceptable salts or solvates for the preparation of a medicament. 12. (Amended) Use of compounds of the formula I according to claim 1 and/or their physiologically acceptable salts or solvates for the preparation of a medicament for combating illnesses which are associated with the serotonin and dopamine neuro-transmitter system and in which high-affinity serotonin receptors (5-HT_{1A} receptors) and/or dopamine D₂ receptors are involved. 13. (Amended) Use of compounds of the formula I according to claim 1 and/or their physiologically acceptable salts or solvates for the preparation of a medicament for combating psychoses, symptoms of Alzheimer's disease, pathological states of anxiety, overexcitement, hyperactivity, attention disorders in children and youths, severe development disorders and disorders of social behaviour with mental retardation, depression, obsessive-compulsive disorders in the narrower (OCD) and broader sense (OCSD), impairment of sexual function, sleep disturbances, disorders in nutrient take-up, and psychiatric symptoms of this type as part of age dementia and dementia of the Alzheimer's type, for reducing defects in cognitive ability, or for the prophylaxis and control of cerebral infarctions (apoplexia cerebri), for the treatment of extrapyramidal motor diseases, for the treatment of side-effects which occur in the treatment of extrapyramidal motor diseases with conventional anti-Parkinson's medicaments, or for the treatment of extrapyramidal symptoms (EPS) induced by neuroleptics.

14. (Amended) Use of compounds of the formula I according to claim 1 and/or their physiologically acceptable salts or solvates for the preparation of a medicament for combating schizophrenia.
